(19) United States
(12) Patent Application Publication
(10) Pub. No.: US 2024/0180906 A1
(43) Pub. Date: Jun. 6, 2024

(54) Combination of Talazoparib and an Anti-Androgen for the Treatment of DDR Gene Mutated Metastatic Castration-Sensitive Prostate Cancer

(71) Applicants: Pfizer Inc., New York, NY (US); Astellas Pharma Inc., Tokyo (JP)

(72) Inventors: Akos Gabor Czibere, Cambridge, MA (US); Dana Ann Kennedy, San Diego, CA (US)

(73) Assignees: Pfizer Inc., New York, NY (US); Astellas Pharma Inc., Tokyo (JP)

(21) Appl. No.: 18/283,634

(22) PCT Filed: Mar. 21, 2022
(86) PCT No.: PCT/IB2022/052536

§ 371 Date: Sep. 22, 2023

Related U.S. Application Data
(63) Continuation of application No. 63/317,368, filed on Mar. 7, 2022.
(60) Provisional application No. 63/282,163, filed on Nov. 22, 2021.
(60) Provisional application No. 63/165,723, filed on Mar. 24, 2021.

(51) Int. Cl. A61K 31/5025; A61K 31/4166; A61K 45/06

(52) U.S. Cl. CPC A61K 31/5025; A61K 31/4166; A61K 45/06

(57) ABSTRACT

This invention relates to combination therapies comprising talazoparib, or a pharmaceutically acceptable salt thereof, and an anti-androgen, or a pharmaceutically acceptable salt thereof, and associated pharmaceutical compositions, methods of treatment, and pharmaceutical uses for the treatment of metastatic castration-sensitive prostate cancer in subjects identified as having at least one DNA damage repair gene mutation.

### FIELD OF THE INVENTION

The present invention relates to combination therapies useful for the treatment of DNA damage repair (DDR) gene mutated metastatic castration-sensitive prostate cancer. In particular, the invention relates to a combination therapy which comprises talazoparib or a pharmaceutically acceptable salt thereof, and an anti-androgen or a pharmaceutically acceptable salt thereof. The invention also relates to associated methods of treatment, pharmaceutical compositions, and pharmaceutical uses.

### BACKGROUND

Prostate cancer is the second leading cause of cancer death in men. Although the incidence of localized disease has begun to decline within the last few years, the number of patients diagnosed with metastatic prostate cancer has increased. Similar to breast cancer, prostate cancer is a hormonally driven disease. Testosterone and other male sex hormones, known collectively as androgens, are key in the growth of both normal prostate and prostate cancer cells. Androgens can fuel the growth of prostate cancer cells by binding to and activating the androgen receptor. The androgen receptor (AR) is an androgen-stimulated transcription factor that is known to play a role in promoting certain cancers, including the development and progression of prostate cancer.

Anti-androgens are thought to suppress androgen activity by a number of different mechanisms. One example of an anti-androgen approved for the treatment of metastatic castration-resistant prostate cancer and metastatic high-risk castration-sensitive prostate cancer is abiraterone acetate (marketed as Zytiga™), a steroidal CY17A1 inhibitor, which is approved in combination with prednisone. One specific class of anti-androgens are androgen receptor inhibitors, also known as androgen receptor antagonists, which are thought to compete with endogenous ligands, androgens, for the androgen receptor. When an antagonist binds to an androgen receptor it is thought to induce a conformational change in the receptor itself that impedes transcription of key androgen regulated genes and therefore inhibits the biological effects of the androgens themselves, such as testosterone and dihydrotestosterone. Enzalutamide (marketed as Xtandi®) is a non-steroidal androgen receptor inhibitor approved for the treatment of castration-resistant prostate cancer and metastatic castration-sensitive prostate cancer.

Metastatic castration-sensitive prostate cancer (mCSPC), also known as metastatic hormone sensitive prostate cancer (mHSPC), is an advanced prostate cancer that has spread beyond the prostate area to another part of the body. mCSPC can be diagnosed either de novo (patients initially present with metastatic disease without prior treatment of localized disease) or following relapse after treatment of localized disease. For decades, the standard of care for advanced prostate cancer, which includes metastatic disease, has been hormonal therapy, also known as androgen deprivation therapy (ADT), which is intended to lower testosterone levels. ADT remains the backbone therapy for the treatment of mCSPC. Several treatments, including docetaxel, abiraterone plus prednisone, enzalutamide, and apalutamide, have each demonstrated clinical benefit when used in conjunction with ADT, or with physical castration, for example bilateral orchiectomy.

Poly (ADP-ribose) polymerase (PARP) engages in the naturally occurring process of DNA repair in a cell. PARP inhibition has been shown to be an effective therapeutic strategy against tumors associated with germline mutation in double-strand DNA repair genes by inducing synthetic lethality (Sonnenblick, A., et al., Nat Rev Clin Oncol., 2015. 12(1), 27-4).

Talazoparib is a potent, orally available PARP inhibitor, which is cytotoxic to human cancer cell lines harboring gene mutations that compromise deoxyribonucleic acid (DNA) repair, an effect referred to as synthetic lethality, and by trapping PARP protein on DNA thereby preventing DNA repair, replication, and transcription.

The compound, talazoparib, which is “(8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one” and “(8S,9R)-5-fluoro-8-(4-fluorophenyl)-9-(1-methyl-1H-1,2,4-triazol-5-yl)-2,7,8,9-tetrahydro-3H-pyrido[4,3,2-de]phthalazin-3-one” (also referred to as “PF-06944076”, “MDV3800”, and “BMN673”) is a PARP inhibitor, having the structure,

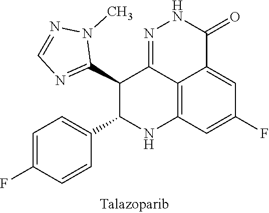 [SMILES: Cn1ncnc1[C@H]1c2n[nH]c(=O)c3cc(F)cc(c23)N[C@@H]1c1ccc(F)cc1]

Talazoparib, and pharmaceutically acceptable salts thereof, including the tosylate salt, are disclosed in International Publication Nos. WO 2010/017055 and WO 2012/054698. Additional methods of preparing talazoparib, and pharmaceutically acceptable salts thereof, including the tosylate salt, are described in International Publication Nos. WO 2011/097602, WO 2015/069851, and WO 2016/019125. Additional methods of treating cancer using talazoparib, and pharmaceutically acceptable salts thereof, including the tosylate salt, are disclosed in International Publication Nos. WO 2011/097334 and WO 2017/075091.

Talazoparib, as a single agent, has demonstrated efficacy, as well as an acceptable toxicity profile in patients with multiple types of solid tumors with DNA repair pathway abnormalities. There are also data supporting the efficacy of talazoparib in combination with chemotherapy in solid tumor types.

Talazoparib (marketed as TALZENNA®) is approved for the treatment of patients with metastatic HER2-negative breast cancer and gBRCA 1/2 mutations. Additionally, the benefit of talazoparib monotherapy is being investigated in docetaxel-pretreated patients with DDR-deficient metastatic castration-resistant prostate cancer in the TALAPRO-1 study (de Bono, J., et al., J Clin Oncol. 2020; 38:5566).

There remains a need for improved therapies for the treatment of cancers. Patients with mCSPC and DDR gene mutations are associated with a poor prognosis. There is a necessity to develop further therapies that will prolong the time to developing castrate resistance and, in turn, will also prolong overall survival. The combinations of the present invention are believed to have one or more advantages, such as improved therapeutic benefits than treatment with either therapeutic agent alone; potential to enable an improved dosing schedule; potential to overcome resistance mechanisms and the like. These, and other advantages of the present invention, are apparent from the description below.

### SUMMARY

Each of the embodiments of the present invention described below may be combined with one or more other embodiments of the present invention described herein which is not inconsistent with the embodiment(s) with which it is combined. In addition, each of the embodiments below describing the invention envisions within its scope the pharmaceutically acceptable salts of the compounds of the invention. Accordingly, the phrase “or a pharmaceutically acceptable salt thereof” is implicit in the description of all compounds described herein.

This invention relates to a method of treating metastatic castration-sensitive prostate cancer in a subject in need thereof, wherein the subject has been identified as having at least one DNA damage repair gene mutation, comprising administering to the subject a combination therapy which comprises talazoparib, or a pharmaceutically acceptable salt thereof, and an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof, wherein radiographic progression-free survival is prolonged as compared to placebo in combination with an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof, wherein overall survival is prolonged as compared to placebo in combination with an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof, wherein occurrence of castration resistance is prolonged as compared to placebo in combination with an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof, wherein time to prostate specific antigen (PSA) progression is prolonged as compared to placebo in combination with an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof, wherein radiographic progression-free survival is prolonged as compared to placebo in combination with an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof, wherein overall survival is prolonged as compared to placebo in combination with an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof, wherein occurrence of castration resistance is prolonged as compared to placebo in combination with an anti-androgen, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof, wherein time to prostate specific antigen (PSA) progression is prolonged as compared to placebo in combination with an anti-androgen, or a pharmaceutically acceptable salt thereof.

In one embodiment of the present invention, the at least one DNA damage repair gene mutation is selected from the group consisting of ATM, ATR, BRCA1, BRCA2, CDK12, CHEK2, FANCA, MLH1, MRE11A, NBN, PALB2, and RAD51C.

In one embodiment of the present invention, the at least one DNA damage repair gene mutation is selected from the group consisting of ATM, BRCA1, and BRCA2.

In one embodiment of the present invention, the at least one DNA damage repair gene mutation is selected from the group consisting of ATR, CDK12, CHEK2, FANCA, MLH1, MRE11A, NBN, PALB2, and RAD51C.

In one embodiment of the present invention, the at least one DNA damage repair gene mutation is ATM; the at least one DNA damage repair gene mutation is ATR; the at least one DNA damage repair gene mutation is BRCA1; the at least one DNA damage repair gene mutation is BRCA2; the at least one DNA damage repair gene mutation is CDK12; the at least one DNA damage repair gene mutation is CHEK2; the at least one DNA damage repair gene mutation is FANCA; the at least one DNA damage repair gene mutation is MLH1; the at least one DNA damage repair gene mutation is MRE11A; the at least one DNA damage repair gene mutation is NBN; the at least one DNA damage repair gene mutation is PALB2; and the at least one DNA damage repair gene mutation is RAD51C.

In one embodiment of the present invention, the subject is treatment naive.

In one embodiment of the present invention, the combination therapy is first-line treatment for metastatic castration-sensitive prostate cancer.

In one embodiment of the present invention, the talazoparib, or a pharmaceutically acceptable salt thereof, is talazoparib tosylate.

In one embodiment of the present invention, the anti-androgen, or a pharmaceutically acceptable salt thereof, is an androgen receptor inhibitor, or a pharmaceutically acceptable salt thereof.

In one embodiment of the present invention, the anti-androgen is selected from the group consisting of:
abiraterone acetate;
enzalutamide;
N-desmethyl enzalutamide;
darolutamide; and
apalutamide,
or a pharmaceutically acceptable salt thereof.

In one embodiment of the present invention, the anti-androgen is enzalutamide, or a pharmaceutically acceptable salt thereof.

In one embodiment of the present invention, the talazoparib, or pharmaceutically acceptable salt thereof, is administered at a daily dosage of about 0.35 mg or about 0.5 mg once daily and the enzalutamide, or a pharmaceutically acceptable salt thereof, is administered at a daily dosage of about 160 mg.

In one embodiment of the present invention, the talazoparib, or pharmaceutically acceptable salt thereof, is administered at a daily dosage of about 0.35 mg once daily and the enzalutamide, or a pharmaceutically acceptable salt thereof, is administered at a daily dosage of about 160 mg.

In one embodiment of the present invention, the talazoparib, or pharmaceutically acceptable salt thereof, is administered at a daily dosage of about 0.5 mg once daily and the enzalutamide, or a pharmaceutically acceptable salt thereof, is administered at a daily dosage of about 160 mg.

In one embodiment of the present invention, the talazoparib, or pharmaceutically acceptable salt thereof, is administered at a daily dosage of about 0.35 mg once daily.

In one embodiment of the present invention, the talazoparib, or pharmaceutically acceptable salt thereof, is administered at a daily dosage of about 0.5 mg once daily.

In one embodiment of the present invention, the enzalutamide, or a pharmaceutically acceptable salt thereof is administered at a daily dosage of about 160 mg.

In one embodiment of the present invention, the talazoparib, or pharmaceutically acceptable salt thereof, and the anti-androgen, or pharmaceutically acceptable salt thereof, are each in an amount that is together effective in treating metastatic castration-sensitive prostate cancer.

In one embodiment of the present invention, the talazoparib, or pharmaceutically acceptable salt thereof, and the anti-androgen, or a pharmaceutically acceptable salt or solvate thereof, are administered concurrently.

In one embodiment of the present invention, a further anti-cancer agent is administered.

In one embodiment of the present invention, the further anti-cancer agent is selected from the group consisting of an anti-tumor agent, an anti-angiogenesis agent, a signal transduction inhibitor, an antiproliferative agent, and androgen deprivation therapy.

In one embodiment of the present invention, the subject is a human.

This invention relates to a method of treating metastatic castration-sensitive prostate cancer in a subject in need thereof, wherein the subject has been identified as having at least one DNA damage repair gene mutation, comprising administering to the subject a combination therapy which comprises talazoparib, or a pharmaceutically acceptable salt thereof, and enzalutamide, or a pharmaceutically acceptable salt thereof.

This invention relates to a method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and enzalutamide, or a pharmaceutically acceptable salt thereof.

In one embodiment of the present invention, the at least one DNA damage repair gene mutation is selected from the group consisting of ATM, ATR, BRCA1, BRCA2, CDK12, CHEK2, FANCA, MLH1, MRE11A, NBN, PALB2, and RAD51C.

In one embodiment of the present invention, the subject is treatment naive.

In one embodiment of the present invention, the talazoparib, or a pharmaceutically acceptable salt thereof, is talazoparib tosylate.

In one embodiment of the present invention, the talazoparib, or pharmaceutically acceptable salt thereof, is administered at a daily dosage of about 0.35 mg or about 0.5 mg once daily and the enzalutamide, or a pharmaceutically acceptable salt thereof, is administered at a daily dosage of about 160 mg.

In one embodiment of the present invention, the talazoparib, or pharmaceutically acceptable salt thereof, is administered at a daily dosage of about 0.35 mg once daily and the enzalutamide, or a pharmaceutically acceptable salt thereof, is administered at a daily dosage of about 160 mg.

In one embodiment of the present invention, the talazoparib, or pharmaceutically acceptable salt thereof, is administered at a daily dosage of about 0.5 mg once daily and the enzalutamide, or a pharmaceutically acceptable salt thereof, is administered at a daily dosage of about 160 mg.

In one embodiment of the present invention, the talazoparib, or pharmaceutically acceptable salt thereof, is administered at a daily dosage of about 0.35 mg once daily.

In one embodiment of the present invention, the talazoparib, or pharmaceutically acceptable salt thereof, is administered at a daily dosage of about 0.5 mg once daily.

In one embodiment of the present invention, the enzalutamide, or a pharmaceutically acceptable salt thereof is administered at a daily dosage of about 160 mg.

In one embodiment of the present invention, the talazoparib, or pharmaceutically acceptable salt thereof, and the anti-androgen, or pharmaceutically acceptable salt thereof, are each in an amount that is together effective in treating metastatic castration-sensitive prostate cancer.

In one embodiment of the present invention, the talazoparib, or pharmaceutically acceptable salt thereof, and the anti-androgen, or a pharmaceutically acceptable salt or solvate thereof, are administered concurrently.

In one embodiment of the present invention, a further anti-cancer agent is administered.

In one embodiment of the present invention, the further anti-cancer agent is selected from the group consisting of an anti-tumor agent, an anti-angiogenesis agent, a signal transduction inhibitor, an antiproliferative agent, and androgen deprivation therapy.

In one embodiment of the present invention, wherein a further anti-cancer agent is administered and the further anti-cancer agent is an androgen deprivation therapy, the androgen deprivation therapy is selected from the group consisting of a luteinizing hormone-releasing hormone agonist, a luteinizing hormone-releasing hormone antagonist, a gonadotropin releasing hormone agonist, a gonadotropin releasing hormone antagonist, and bilateral orchiectomy.

In one embodiment of the present invention, the subject is a human.

### DETAILED DESCRIPTION

The present invention may be understood more readily by reference to the following detailed description of the preferred embodiments of the invention. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The term “about” when used to modify a numerically defined parameter (e.g., the dose of a talazoparib, or a pharmaceutically acceptable salt thereof, the dose of an anti-androgen, the dose of an androgen receptor inhibitor and the like) means that the parameter may vary by as much as 10% above or below the stated numerical value for that parameter. For example, a dose of about 1.0 mg once daily should be understood to mean that the dose may vary between 0.9 mg once daily and 1.1 mg once daily.

As used herein, the term “anti-androgen” and “anti-androgens” shall be taken to mean compounds which prevent androgens, for example testosterone and dihydrotestosterone (DHT) and the like, from mediating their biological effects in the body. Anti-androgens may act by one or more of the following hormonal mechanisms of action such as blocking and/or inhibiting and/or modulating the androgen receptor (AR); inhibiting androgen production; suppressing androgen production; degrading the AR, inhibiting nuclear translocation, inhibiting binding of the AR to nuclear DNA, and the like. Anti-androgens include, but are not limited to, steroidal androgen receptor inhibitors (for example, cyproterone acetate, spironolactone, megestrol acetate, chlormadinone acetate, oxendolone, and osaterone acetate), non-steroidal androgen receptor inhibitors (for example, enzalutamide, bicalutamide, nilutamide, flutamide, topilutamide), androgen synthesis inhibitors, androgen receptor degraders and the like.

“Angiogenesis” as used herein refers to blood vessel formation. Tumor angiogenesis is the growth of new blood vessels that tumors need to grow. This process is caused by the release of chemicals by the tumor and by host cells near the tumor.

The terms “abnormal cell growth” and “hyperproliferative disorder” are used interchangeably in this application.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous).

“Apoptosis” as used herein refers to the death of cells that occurs as a normal and controlled part of an organism's growth or development. Apoptosis is a type of cell death in which a series of molecular steps in a cell lead to its death. Apoptosis is one method the body uses to get rid of unneeded or abnormal cells. The process of apoptosis may be blocked in cancer cells.

The terms “cancer”, “cancerous”, and “malignant” refer to or describe the physiological condition in mammals that is typically characterized by unregulated cell growth. As used herein “cancer” refers to any malignant and/or invasive growth or tumor caused by abnormal cell growth. As used herein “cancer” refers to solid tumors. The term “cancer” includes, but is not limited to, a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from latter one. An example of cancer for purposes of the present invention includes metastatic castration-sensitive prostate cancer.

As used herein, the term “metastatic” as the term relates to cancer, such as a prostate cancer, is documented by positive bone scan (for bone disease) or metastatic lesions on Computerized Tomography (CT) or Magnetic Resonance Imaging (MRI) scan (for soft tissue disease).

As used herein, the term “metastatic castration-sensitive prostate cancer (mCSPC)”, also known as “metastatic hormone sensitive prostate cancer (mHSPC)”, is an advanced prostate cancer that has spread beyond the prostate area to another part of the body. mCSPC can be diagnosed either de novo (patients initially present with metastatic disease without prior treatment of localized disease) or following relapse after treatment of localized disease. mCSPC may include and one or more of the following: 1) de novo mCSPC; 2) relapsed mCSPC; 3) high volume disease; (high volume disease is defined as the presence of visceral metastases or ≥4 bone lesions with ≥1 beyond the vertebral bodies and pelvis); 4) low volume disease; 5) BRCA mutational status; and 6) non-BRCA mutational status.

The term “patient” or “subject” refers to any single subject for which therapy is desired or that is participating in a clinical trial, epidemiological study or used as a control, including humans and mammalian veterinary patients such as cattle, horses, dogs and cats. In certain preferred embodiments, the subject is a human. A “patient” or “subject” according to the combination of this invention may have: 1) histologically or cytologically confirmed adenocarcinoma of the prostate without neuroendocrine differentiation, small cell or signet cell features; 2) histologically or cytologically confirmed adenocarcinoma of the prostate without small cell or signet cell features; 3) metastatic castration-sensitive prostate cancer; 4) DNA damage repair (DDR) deficiency as assessed centrally by a next-generation sequencing (NGS) biomarker mutation panel that contains DDR genes likely to sensitize to PARP inhibition, such as the FoundationOne® test or the FoundationOne® Liquid CDx test; 5) surgically or medically castrated, with serum testosterone ≤50 ng/dL (≤1.73 nmol/L) at screening; 6) ongoing androgen deprivation therapy with a gonadotropin releasing hormone (GnRH) agonist or antagonist for patients who have not undergone bilateral orchiectomy; 7) metastatic disease in bone documented on bone scan or in soft tissue documented on CT/MRI scan; and 8) Eastern Cooperative Oncology Group (ECOG) performance status 0 or 1.

The term “treat” or “treating” a cancer as used herein means to administer a combination therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (200)). In some embodiments, the treatment achieved by a combination of the invention is any of the partial response (PR), complete response (CR), overall response (OR), objective response rate (ORR), progression free survival (PFS), radiographic PFS (rPFS), and overall survival (OS). rPFS indicates the time from the date of randomization to first objective evidence of radiographic progression as assessed in soft tissue per RECIST 1.1 or in bone (upon subsequent confirmation) per PCWG3 guidelines by investigator, or death, whichever occurs first. OS refers to a prolongation in life expectancy as compared to naïve or untreated subjects or patients. In some embodiments, response to a combination of the invention is any of PR, CR, PFS, ORR, OR or OS. Response to a combination of the invention, including duration of soft tissue response, is assessed using Response Evaluation Criteria in Solid Tumors version 1.1 (RECIST 1.1) response criteria. In some embodiments, the treatment achieved by a combination of the invention is measured by the time to prostate-specific antigen (PSA) progression, the time to initiation of cytotoxic chemotherapy and the proportion of patients with PSA response greater than or equal to 50%. The treatment regimen for a combination of the invention that is effective to treat a cancer patient may vary according to factors such as the disease state, age, and weight of the patient, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as, but not limited to, the Cox log-rank test, the Cochran-Mantel-Haenszel log-rank test, the Student's t-test, the chi2-test, the U-test according to Mann and Whitney, the Kruskal-Wallis test (H-test), Jonckheere-Terpstrat-test and the Wilcon on-test. The term “treatment” also encompasses in vitro and ex vivo treatment, e.g., of a cell, by a reagent, diagnostic, binding compound, or by another cell.

The terms “treatment regimen”, “dosing protocol” and “dosing regimen” are used interchangeably to refer to the dose and timing of administration of each therapeutic agent in a combination of the invention.

“Ameliorating” means a lessening or improvement of one or more symptoms as compared to not administering a therapeutic agent of a method or regimen of the invention. “Ameliorating” also includes shortening or reduction in duration of a symptom.

As used herein, an “effective dosage” or “effective amount” of drug, compound or pharmaceutical composition is an amount sufficient to effect any one or more beneficial or desired, including biochemical, histological and/or behavioural symptoms, of the disease, its complications and intermediate pathological phenotypes presenting during development of the disease. For therapeutic use, an “effective amount” refers to that amount of a compound being administered which will relieve to some extent one or more of the symptoms of the disorder being treated. In reference to the treatment of cancer, an effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) relieving to some extent (or, preferably, eliminating) one or more signs or symptoms associated with the cancer, (5) decreasing the dose of other medications required to treat the disease, and/or (6) enhancing the effect of another medication, and/or delaying the progression of the disease of patients. An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another drug, compound or pharmaceutical composition.

The terms “taxane”, “taxanes”, and “taxane based chemotherapy” are used interchangeably to refer to a class of chemotherapeutic agents, including, but not limited to paclitaxel, docetaxel and cabazitaxel.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Examples of solid tumors are sarcomas, carcinomas, and lymphomas. Leukaemia's (cancers of the blood) generally do not form solid tumors (National Cancer Institute, Dictionary of Cancer Terms).

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CR or MRI scans.

A “non standard clinical dosing regimen” as used herein, refers to a regimen for administering a substance, agent, compound or composition, which is different to the amount, dose or schedule typically used for that substance, agent, compound or composition in a clinical setting. A “non-standard clinical dosing regimen”, includes a “non-standard clinical dose” or a “non-standard dosing schedule”.

A “low dose amount regimen” as used herein refers to a dosing regimen where one or more of the substances, agents, compounds or compositions in the regimen is dosed at a lower amount or dose than typically used in a clinical setting for that agent, for example when that agent is dosed as a singleton therapy.

### Anti-Androgens

Embodiments of the present invention relate to anti-androgens, or a pharmaceutically acceptable salt thereof. Embodiments of the present invention also relate to anti-androgens, or a pharmaceutically acceptable salt thereof.

In one embodiment, the anti-androgen is a compound which degrades the androgen receptor.

In one embodiment, the anti-androgen is a compound which inhibits and/or suppresses the production of androgens.

In one embodiment, the anti-androgen is abiraterone, or a pharmaceutically acceptable salt thereof, such as abiraterone acetate (marketed as Zytiga™), a steroidal CY17A1 inhibitor which is disclosed in U.S. Pat. No. 5,604,213 which published on 18 Feb. 1997, the contents of which are incorporated herein by reference.

In one embodiment the anti-androgen is an androgen receptor inhibitor, or a pharmaceutically acceptable salt thereof. In one embodiment the anti-androgen is an androgen receptor inhibitor, or a pharmaceutically acceptable salt thereof. Androgen receptor inhibitors include, but are not limited to, non-steroidal small molecule androgen receptor inhibitors, or pharmaceutically acceptable salts thereof. Androgen receptor inhibitors can be determined by methods known to those of skilled in the art, for example using in vitro assays and/or cellular ligand binding assays and/or gene expression assays such as those disclosed in Tran C., et al., Science, 2009, 324, 787-790.

Examples of specific androgen receptor inhibitors that are useful in the present invention include those disclosed in International patent application PCT/US2006/011417, which published on 23^{rd} Nov. 2006 as WO 2006/124118, the contents of which are included herein by reference, or a pharmaceutically acceptable salt thereof. Specific androgen receptor inhibitors disclosed therein useful as the androgen receptor inhibitor for the present invention include, but are not limited to, androgen receptor inhibitors selected from the group consisting of:

RD7; RD8; RD10; RD35; RD36; RD37; RD57; RD58; RD90; RD91; RD92; RD93; RD94; RD95; RD96; RD97; RD100; RD102; RD119; RD120; RD130; RD131; RD145; RD152; RD153; RD163; RD162; RD162′; RD162″; RD168; RD169; and RD170

or a pharmaceutically acceptable salt thereof.

Other examples of specific androgen receptor inhibitors that are useful in the present invention include those disclosed in International patent application PCT/US2007/007854, which published on 8^{th} Nov. 2007 as WO 2007/127010, the contents of which are included herein by reference, or a pharmaceutically acceptable salt thereof.

Other examples of specific androgen receptor inhibitors that are useful in the present invention include those disclosed in International patent application PCT/US2008/012149, which published on 30^{th} Apr. 2009 as WO 2009/055053, the contents of which are included herein by reference, or a pharmaceutically acceptable salt thereof.

Other examples of specific androgen receptor inhibitors that are useful in the present invention include those disclosed in International patent application PCT/US2007/007485, which published on 8^{th} Nov. 2007 as WO 2007/126765, the contents of which are included herein by reference. Examples of specific androgen receptor inhibitors disclosed therein useful as the androgen receptor inhibitor for the present invention include, but are not limited to, androgen receptor inhibitors selected from the group consisting of:

A51; and A52

or a pharmaceutically acceptable salt thereof.

Other examples of specific androgen receptor inhibitors that are useful in the present invention include those disclosed in International patent application PCT/US2010/030581, which published on 14^{th} Oct. 2010 as WO 2010/118354, the contents of which are included herein by reference, or a pharmaceutically acceptable salt thereof.

Other examples of specific androgen receptor inhibitors that are useful in the present invention include those disclosed in International patent application PCT/US2010/051770, which published on 14^{th} Apr. 2011 as WO 2011/044327, the contents of which are included herein by reference, or a pharmaceutically acceptable salt thereof.

Other examples of specific androgen receptor inhibitors that are useful in the present invention include those disclosed in International patent application PCT/US2010/025283, which published on 2^{nd} Sep. 2010 as WO 2010/099238, the contents of which are included herein by reference. Examples of specific androgen receptor inhibitors disclosed therein useful as the androgen receptor inhibitor for the present invention include, but are not limited to, androgen receptor inhibitors selected from the group consisting of:

MII

or a pharmaceutically acceptable salt thereof.

Other examples of specific androgen receptor inhibitors that are useful in the present invention include those disclosed in International patent application PCT/FI2010/000065, which published on 5^{th} May 2011 as WO 2011/051540, the contents of which are included herein by reference.

Other examples of specific androgen receptor inhibitors that are useful in the present invention include those disclosed in U.S. Pat. No. 4,636,505, published on 13^{th} Jan. 1987, the contents of which are included herein by reference.

In one embodiment, the androgen receptor inhibitor useful in the present invention is enzalutamide:

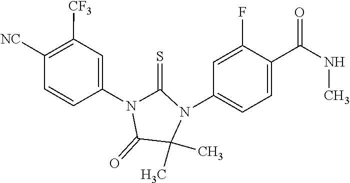 [SMILES: CNC(=O)c1ccc(N2C(=S)N(c3ccc(C#N)c(C(F)(F)F)c3)C(=O)C2(C)C)cc1F]

or a pharmaceutically acceptable salt thereof, also known as RD162′; 4-[3-[4-cyano-3-(trifluoromethyl)phenyl]-5,5-dimethyl-4-oxo-2-thioxo-1-imidazolidinyl]-2-fluoro-N-methylbenzamide; or 4-{3-[4-cyano-3-(trifluoromethyl)phenyl]-5,5-dimethyl-4-oxo-2-sulfanylideneimidazolidin-1-yl}-2-fluoro-N-methylbenzamide; which is disclosed in PCT/US2006/011417, which published on 23^{rd} Nov. 2006 as WO 2006/124118, the contents of which are included herein by reference.

In one embodiment, the androgen receptor inhibitor useful in the present invention is N-desmethyl enzalutamide:

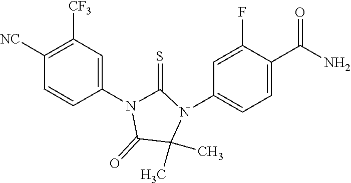 [SMILES: CC1(C)C(=O)N(c2ccc(C#N)c(C(F)(F)F)c2)C(=S)N1c1ccc(C(N)=O)c(F)c1]

or a pharmaceutically acceptable salt thereof, also known as 4-[3-[4-cyano-3-(trifluoromethyl)phenyl]-5,5-dimethyl-4-oxo-2-thioxoimidazolidin-1-yl]-2-fluorobenzamide; or MII; which is disclosed in PCT/US2010/025283, which published on 2^{nd} Sep. 2010 as WO 2010/099238, the contents of which are included herein by reference.

In one embodiment, the androgen receptor inhibitor useful in the present invention is apalutamide:

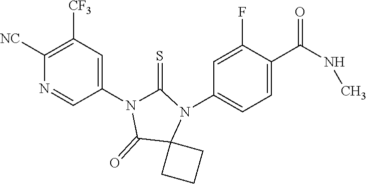 [SMILES: CNC(=O)c1ccc(N2C(=S)N(c3cnc(C#N)c(C(F)(F)F)c3)C(=O)C23CCC3)cc1F]

or a pharmaceutically acceptable salt thereof, also known as ARN-509; or 4-{7-[6-cyano-5-(trifluoromethyl)pyridine-3-yl]-8-oxo-6-thioxo-5,7-diazaspiro[3,4]octan-5yl}-2-fluoro-N-methylbenzamide; which is disclosed in PCT/US2007/007485, which published on 8^{th} Nov. 2007 as WO 2007/126765, the contents of which are included herein by reference. In one embodiment, the androgen receptor inhibitor useful in the present invention is a pharmacologically active metabolite of apalutamide, or a pharmaceutically acceptable salt thereof.

In one embodiment, the androgen receptor inhibitor useful in the present invention is darolutamide:

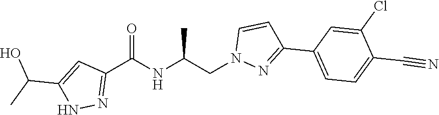 [SMILES: CC(O)c1cc(C(=O)N[C@@H](C)Cn2ccc(-c3ccc(C#N)c(Cl)c3)n2)n[nH]1]

or a pharmaceutically acceptable salt thereof, also known as N-[(2S)-1-[3-(3-chloro-4-cyanophenyl)-1H-pyrazol-1-yl]propan-2-yl]-5-(1-hydroxyethyl)-1H-pyrazole-3-carboxamide which is disclosed in PCT/F12010/000065, which published on 5^{th} May 2011 as WO 2011/051540, the contents of which are included herein by reference.

In one embodiment, the androgen receptor inhibitor useful in the present invention is bicalutamide:

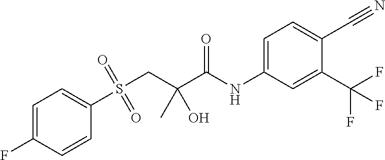 [SMILES: CC(O)(CS(=O)(=O)c1ccc(F)cc1)C(=O)Nc1ccc(C#N)c(C(F)(F)F)c1]

or a pharmaceutically acceptable salt thereof, marketed as Casodex™, which is disclosed in U.S. Pat. No. 4,636,505, published on 13^{th} Jan. 1987, the contents of which are included herein by reference.

In one embodiment, the androgen receptor inhibitor useful in the present invention is nilutamide, or a pharmaceutically acceptable salt thereof.

In one embodiment, the androgen receptor inhibitor useful in the present invention is flutamide, or a pharmaceutically acceptable salt thereof.

Preferred androgen receptor inhibitors useful for the present invention are selected from the group consisting of:
enzalutamide;
N-desmethyl enzalutamide;
darolutamide; and
apalutamide;
or a pharmaceutically acceptable salt thereof.

More preferred androgen receptors inhibitors useful for the present invention is enzalutamide, or a pharmaceutically acceptable salt thereof. More preferably the androgen receptor inhibitor is enzalutamide.

In one embodiment the anti-androgen is administered in combination with androgen deprivation therapy.

In one embodiment the androgen deprivation therapy is orchiectomy.

In one embodiment the androgen deprivation therapy is bilateral orchiectomy.

In one embodiment the anti-androgen is administered in combination with androgen deprivation therapy, which androgen deprivation therapy is selected from the group consisting of a luteinizing hormone-releasing hormone (LHRH) agonist, a LHRH antagonist, a gonadotropin releasing hormone (GnRH) agonist and a GnRH antagonist.

In one embodiment the androgen deprivation therapy is selected from the group consisting of leuprolide (also known as leuprorelin, for example Lupron or Eligardor Viadur and the like); buserelin (for example Suprefact); gonadorelin; goserelin (for example Zoladex); histrelin (for example Vantas); nafarelin; triptorelin (for example Trelstar); deslorelin; fertirelin; abarelix (for example Plenaxis); cetrorelix; degarelix (for example Firmagon); ganirelix; ozarelix; elagolix (for example Orilissa); relugolix; and linzagolix.

In one embodiment the androgen deprivation therapy is leuprolide.

In one embodiment the androgen deprivation therapy is goserelin.

In one embodiment the androgen deprivation therapy is degarelix.

In one embodiment the androgen deprivation therapy is relugolix.

In one embodiment the anti-androgen is enzalutamide and the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix. In one embodiment the anti-androgen is enzalutamide and the androgen deprivation therapy is selected from the group consisting of leuprolide, goserelin, degarelix and relugolix.

In one embodiment the anti-androgen is N-desmethyl enzalutamide and the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix. In one embodiment the anti-androgen is N-desmethyl enzalutamide and the androgen deprivation therapy is selected from the group consisting of leuprolide, goserelin, degarelix and relugolix.

In one embodiment the anti-androgen is apalutamide and the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix. In one embodiment the anti-androgen is apalutamide and the androgen deprivation therapy is selected from the group consisting of leuprolide, goserelin, degarelix and relugolix.

In one embodiment the anti-androgen is abiraterone, preferably abiraterone acetate, and the androgen deprivation therapy is selected from the group consisting of leuprolide; buserelin gonadorelin; goserelin; histrelin; nafarelin; triptorelin; deslorelin; fertirelin; abarelix; cetrorelix; degarelix; ganirelix; ozarelix; elagolix; relugolix; and linzagolix. In one embodiment the anti-androgen is abiraterone, preferably abiraterone acetate, and the androgen deprivation therapy is selected from the group consisting of leuprolide, goserelin, degarelix and relugolix.

Unless indicated otherwise, all references herein to the anti-androgens and androgen receptor inhibitors includes references to salts, solvates, hydrates and complexes thereof, and to solvates, hydrates and complexes of salts thereof, including polymorphs, stereoisomers, and isotopically labeled versions thereof.

### Therapeutic Methods and Uses

The methods and combination therapies of the present invention are useful for treating cancer.

In one embodiment, this invention relates to a method of treating metastatic castration-sensitive prostate cancer in a subject in need thereof, wherein the subject has been identified as having at least one DNA damage repair gene mutation, comprising administering to the subject a combination therapy which comprises talazoparib, or a pharmaceutically acceptable salt thereof, and an anti-androgen, or a pharmaceutically acceptable salt thereof.

In another aspect, this invention relates to a method of treating metastatic castration-sensitive prostate cancer in a subject in need thereof, wherein the subject has been identified as having at least one DNA damage repair gene mutation, comprising administering to the subject a combination therapy which comprises talazoparib, or a pharmaceutically acceptable salt thereof, and an androgen receptor inhibitor, or a pharmaceutically acceptable salt thereof.

In another aspect, this invention relates to a method of treating metastatic castration-sensitive prostate cancer in a subject in need thereof, wherein the subject has been identified as having at least one DNA damage repair gene mutation, comprising administering to the subject a combination therapy which comprises talazoparib, or a pharmaceutically acceptable salt thereof, and enzalutamide, or a pharmaceutically acceptable salt thereof.

In another aspect, this invention relates to talazoparib, or a pharmaceutically acceptable salt thereof, for use in the treatment of metastatic castration-sensitive prostate cancer in a subject in need thereof, wherein the subject has been identified as having at least one DNA damage repair gene mutation, wherein the talazoparib, or a pharmaceutically acceptable salt thereof, is used in combination with an anti-androgen, or a pharmaceutically acceptable salt thereof.

In another aspect, this invention relates to talazoparib, or a pharmaceutically acceptable salt thereof, for use in the treatment of metastatic castration-sensitive prostate cancer in a subject in need thereof, wherein the subject has been identified as having at least one DNA damage repair gene mutation, wherein the talazoparib, or a pharmaceutically acceptable salt thereof, is used in combination with an androgen receptor inhibitor, or a pharmaceutically acceptable salt thereof.

In another aspect, this invention relates to talazoparib, or a pharmaceutically acceptable salt thereof, for use in the treatment of metastatic castration-sensitive prostate cancer in a subject in need thereof, wherein the subject has been identified as having at least one DNA damage repair gene mutation, wherein the talazoparib, or a pharmaceutically acceptable salt thereof, is used in combination with enzalutamide, or a pharmaceutically acceptable salt thereof.

In another aspect, this invention relates to an anti-androgen, or a pharmaceutically acceptable salt thereof, for use in the treatment of metastatic castration-sensitive prostate cancer in a subject in need thereof, wherein the subject has been identified as having at least one DNA damage repair gene mutation, wherein the anti-androgen is used in combination with talazoparib, or a pharmaceutically acceptable salt thereof.

In another aspect, this invention relates to an androgen receptor inhibitor, or a pharmaceutically acceptable salt thereof, for use in the treatment of metastatic castration-sensitive prostate cancer in a subject in need thereof, wherein the subject has been identified as having at least one DNA damage repair gene mutation, wherein the androgen receptor inhibitor is used in combination with talazoparib, or a pharmaceutically acceptable salt thereof.

In another aspect, this invention relates to enzalutamide, or a pharmaceutically acceptable salt thereof, for use in the treatment of metastatic castration-sensitive prostate cancer in a subject in need thereof, wherein the subject has been identified as having at least one DNA damage repair gene mutation, wherein enzalutamide is used in combination with talazoparib, or a pharmaceutically acceptable salt thereof.

In another aspect, this invention relates to a combination of talazoparib, or a pharmaceutically acceptable salt thereof, and an anti-androgen, or a pharmaceutically acceptable salt thereof, for use in the treatment of metastatic castration-sensitive prostate cancer in a subject in need thereof, wherein the subject has been identified as having at least one DNA damage repair gene mutation.

In another aspect, this invention relates to a combination of talazoparib, or a pharmaceutically acceptable salt thereof, and an androgen receptor inhibitor, or a pharmaceutically acceptable salt thereof, for use in the treatment of metastatic castration-sensitive prostate cancer in a subject in need thereof, wherein the subject has been identified as having at least one DNA damage repair gene mutation.

In another aspect, this invention relates to a combination of talazoparib, or a pharmaceutically acceptable salt thereof, and enzalutamide, or a pharmaceutically acceptable salt thereof, for use in the treatment of metastatic castration-sensitive prostate cancer in a subject in need thereof, wherein the subject has been identified as having at least one DNA damage repair gene mutation.

In another aspect, this invention relates to the use of talazoparib, or a pharmaceutically acceptable salt thereof, and an anti-androgen, or a pharmaceutically acceptable salt thereof, in the manufacture of a medicament for the treatment of metastatic castration-sensitive prostate cancer in a subject in need thereof, wherein the subject has been identified as having at least one DNA damage repair gene mutation.

In another aspect, this invention relates to the use of talazoparib, or a pharmaceutically acceptable salt thereof, and an androgen receptor inhibitor, or a pharmaceutically acceptable salt thereof, in the manufacture of a medicament for the treatment of metastatic castration-sensitive prostate cancer in a subject in need thereof, wherein the subject has been identified as having at least one DNA damage repair gene mutation.

In another aspect, this invention relates to the use of talazoparib, or a pharmaceutically acceptable salt thereof, and enzalutamide, or a pharmaceutically acceptable salt thereof, in the manufacture of a medicament for the treatment of metastatic castration-sensitive prostate cancer in a subject in need thereof, wherein the subject has been identified as having at least one DNA damage repair gene mutation.

In another aspect, this invention relates to a pharmaceutical composition comprising talazoparib, or a pharmaceutically acceptable salt thereof, and a pharmaceutically acceptable carrier for use in the treatment of metastatic castration-sensitive prostate cancer in a subject in need thereof, wherein the subject has been identified as having at least one DNA damage repair gene mutation, wherein the pharmaceutical composition comprising the talazoparib, or a pharmaceutically acceptable salt thereof, is used in combination with a pharmaceutical composition comprising an anti-androgen, or a pharmaceutically acceptable salt thereof, and a pharmaceutically acceptable carrier.

In another aspect, this invention relates to a pharmaceutical composition comprising talazoparib, or a pharmaceutically acceptable salt thereof, and a pharmaceutically acceptable carrier for use in the treatment of metastatic castration-sensitive prostate cancer in a subject in need thereof, wherein the subject has been identified as having at least one DNA damage repair gene mutation, wherein the pharmaceutical composition comprising the talazoparib, or a pharmaceutically acceptable salt thereof, is used in combination with a pharmaceutical composition comprising an androgen receptor inhibitor, or a pharmaceutically acceptable salt thereof, and a pharmaceutically acceptable carrier.

In another aspect, this invention relates to a pharmaceutical composition comprising talazoparib, or a pharmaceutically acceptable salt thereof, and a pharmaceutically acceptable carrier for use in the treatment of metastatic castration-sensitive prostate cancer in a subject in need thereof, wherein the subject has been identified as having at least one DNA damage repair gene mutation, wherein the pharmaceutical composition comprising the talazoparib, or a pharmaceutically acceptable salt thereof, is used in combination with a pharmaceutical composition comprising enzalutamide, or a pharmaceutically acceptable salt thereof, and a pharmaceutically acceptable carrier.

In another aspect, this invention relates to a pharmaceutical composition comprising an anti-androgen, or a pharmaceutically acceptable salt thereof, and a pharmaceutically acceptable carrier for use in the treatment of metastatic castration-sensitive prostate cancer in a subject in need thereof, wherein the subject has been identified as having at least one DNA damage repair gene mutation, wherein the pharmaceutical composition comprising the anti-androgen is used in combination with a pharmaceutical composition comprising talazoparib, or a pharmaceutically acceptable salt thereof, and a pharmaceutically acceptable carrier.

In another aspect, this invention relates to a pharmaceutical composition comprising an androgen receptor inhibitor, or a pharmaceutically acceptable salt thereof, and a pharmaceutically acceptable carrier for use in the treatment of metastatic castration-sensitive prostate cancer in a subject in need thereof, wherein the subject has been identified as having at least one DNA damage repair gene mutation, wherein the pharmaceutical composition comprising the androgen receptor inhibitor is used in combination with a pharmaceutical composition comprising talazoparib, or a pharmaceutically acceptable salt thereof, and a pharmaceutically acceptable carrier.

In another aspect, this invention relates to a pharmaceutical composition comprising enzalutamide, or a pharmaceutically acceptable salt thereof, and a pharmaceutically acceptable carrier for use in the treatment of metastatic castration-sensitive prostate cancer in a subject in need thereof, wherein the subject has been identified as having at least one DNA damage repair gene mutation, wherein the pharmaceutical composition comprising the enzalutamide is used in combination with a pharmaceutical composition comprising talazoparib, or a pharmaceutically acceptable salt thereof, and a pharmaceutically acceptable carrier.

In another aspect, this invention relates to a pharmaceutical composition comprising talazoparib, or a pharmaceutically acceptable salt thereof, and an anti-androgen, or a pharmaceutically acceptable salt thereof, and a pharmaceutically acceptable carrier, for use in the treatment of metastatic castration-sensitive prostate cancer in a subject in need thereof, wherein the subject has been identified as having at least one DNA damage repair gene mutation.

In another aspect, this invention relates to a pharmaceutical composition comprising talazoparib, or a pharmaceutically acceptable salt thereof, and an androgen receptor inhibitor, or a pharmaceutically acceptable salt thereof, and a pharmaceutically acceptable carrier, for use in the treatment of metastatic castration-sensitive prostate cancer in a subject in need thereof, wherein the subject has been identified as having at least one DNA damage repair gene mutation.

In another aspect, this invention relates to a pharmaceutical composition comprising talazoparib, or a pharmaceutically acceptable salt thereof, and enzalutamide, or a pharmaceutically acceptable salt thereof, and a pharmaceutically acceptable carrier, for use in the treatment of metastatic castration-sensitive prostate cancer in a subject in need thereof, wherein the subject has been identified as having at least one DNA damage repair gene mutation.

In one embodiment of any of the methods, uses or pharmaceutical compositions of the invention, radiographic progression-free survival is prolonged as compared to placebo in combination with an anti-androgen, or a pharmaceutically acceptable salt thereof.

In one embodiment of any of the methods, uses or pharmaceutical compositions of the invention, radiographic progression-free survival is prolonged as compared to placebo in combination with an androgen receptor inhibitor, or a pharmaceutically acceptable salt thereof.

In one embodiment of any of the methods, uses or pharmaceutical compositions of the invention, radiographic progression-free survival is prolonged as compared to placebo in combination with enzalutamide, or a pharmaceutically acceptable salt thereof.

In one embodiment of any of the methods, uses or pharmaceutical compositions of the invention, overall survival is prolonged as compared to placebo in combination with an anti-androgen, or a pharmaceutically acceptable salt thereof.

In one embodiment of any of the methods, uses or pharmaceutical compositions of the invention, overall survival is prolonged as compared to placebo in combination with an androgen receptor inhibitor, or a pharmaceutically acceptable salt thereof.

In one embodiment of any of the methods, uses or pharmaceutical compositions of the invention, overall survival is prolonged as compared to placebo in combination with enzalutamide, or a pharmaceutically acceptable salt thereof.

In one embodiment of any of the methods, uses or pharmaceutical compositions of the invention, occurrence of castration resistance is prolonged as compared to placebo in combination with an anti-androgen, or a pharmaceutically acceptable salt thereof.

In one embodiment of any of the methods, uses or pharmaceutical compositions of the invention, occurrence of castration resistance is prolonged as compared to placebo in combination with an androgen receptor inhibitor, or a pharmaceutically acceptable salt thereof.

In one embodiment of any of the methods, uses or pharmaceutical compositions of the invention, occurrence of castration resistance is prolonged as compared to placebo in combination with enzalutamide, or a pharmaceutically acceptable salt thereof.

In one embodiment of any of the methods, uses or pharmaceutical compositions of the invention, time to prostate specific antigen (PSA) progression is prolonged as compared to placebo in combination with an anti-androgen, or a pharmaceutically acceptable salt thereof.

In one embodiment of any of the methods, uses or pharmaceutical compositions of the invention, time to prostate specific antigen (PSA) progression is prolonged as compared to placebo in combination with an androgen receptor inhibitor, or a pharmaceutically acceptable salt thereof.

In one embodiment of any of the methods, uses or pharmaceutical compositions of the invention, time to prostate specific antigen (PSA) progression is prolonged as compared to placebo in combination with enzalutamide, or a pharmaceutically acceptable salt thereof.

The methods and uses of the present invention are directed to a subject: 1) in need of treatment for metastatic castration-sensitive prostate cancer, and 2) identified as having at least one DNA damage repair gene mutation. Mutational status for subjects may be determined by testing for the presence of mutations in defined DDR genes likely to sensitize to PARP inhibition using the FoundationOne® Liquid CDx (Foundation Medicine, Inc., Cambridge, MA) test that includes a DDR gene panel consisting of 12 genes, including ATM, ATR, BRCA1, BRCA2, CDK12, CHEK2, FANCA, MLH1, MRE11A, NBN, PALB2, and RAD51C. Genomic screening to identify alterations in DDR genes may also be performed on tumor tissue using the FoundationOne® CDx (Foundation Medicine, Inc., Cambridge, MA) test. Alterations in DDR genes may also be performed by any suitable validated next-generation sequencing assay.

In one embodiment of any of the methods, uses or pharmaceutical compositions of the invention, the at least one DNA damage repair gene mutation is selected from the group consisting of ATM, ATR, BRCA1, BRCA2, CDK12, CHEK2, FANCA, MLH1, MRE11A, NBN, PALB2, and RAD51C.

In one embodiment of any of the methods, uses or pharmaceutical compositions of the invention, the at least one DNA damage repair gene mutation is selected from the group consisting of ATM, BRCA1, and BRCA2.

In one embodiment of any of the methods, uses or pharmaceutical compositions of the invention, the at least one DNA damage repair gene mutation is selected from the group consisting of ATR, CDK12, CHEK2, FANCA, MLH1, MRE11A, NBN, PALB2, and RAD51C.

In one embodiment of any of the methods, uses or pharmaceutical compositions of the invention, the at least one DNA damage repair gene mutation is ATM; the at least one DNA damage repair gene mutation is ATR; the at least one DNA damage repair gene mutation is BRCA1; the at least one DNA damage repair gene mutation is BRCA2; the at least one DNA damage repair gene mutation is CDK12; the at least one DNA damage repair gene mutation is CHEK2; the at least one DNA damage repair gene mutation is FANCA; the at least one DNA damage repair gene mutation is MLH1; the at least one DNA damage repair gene mutation is MRE11A; the at least one DNA damage repair gene mutation is NBN; the at least one DNA damage repair gene mutation is PALB2; and the at least one DNA damage repair gene mutation is RAD51C.

In one embodiment of the present invention, the subject is treatment naive.

In one embodiment of any of the methods, uses or pharmaceutical compositions of the invention, the talazoparib is talazoparib tosylate.

In one embodiment of any of the methods, uses or pharmaceutical compositions of the invention, the subject is a mammal.

In one embodiment of any of the methods or pharmaceutical compositions of the invention, the subject is a human.

In one embodiment, the cancer is metastatic castration-sensitive prostate cancer, also known as metastatic hormone sensitive prostate cancer. Hormone sensitive prostate cancer is usually characterised by histologically or cytologically confirmed adenocarcinoma of the prostate which is still responsive to androgen deprivation therapy.

In one embodiment, the cancer is metastatic castration-sensitive prostate cancer and the subject is treatment naïve.

In one embodiment, the cancer is metastatic castration-sensitive prostate cancer and the subject has received prior treatment with androgen deprivation therapy such as, but not limited to, luteinizing hormone-releasing hormone (LHRH) agonist or LHRH antagonist, or a gonadotropin-releasing hormone (GnRH) agonist, GnRH antagonist, or bilateral orchiectomy. In one embodiment, the cancer is metastatic castration-sensitive prostate cancer and the subject has received prior treatment with androgen deprivation therapy such as, but not limited to, luteinizing hormone-releasing hormone (LHRH) agonist or LHRH antagonist, or a gonadotropin-releasing hormone (GnRH) agonist or GnRH antagonist. In some embodiments, the GnRH agonist is selected from the group consisting of leuprolide, buserelin, nafarelin, histrelin, goserelin, or deslorelin. In some embodiments the androgen deprivation therapy is leuprolide. In some embodiments the androgen deprivation therapy is goserelin. In some embodiments the androgen deprivation therapy is degarelix. In some embodiments the androgen deprivation therapy is relugolix.

In one embodiment, the cancer is metastatic castration-sensitive prostate cancer and the subject continues with maintenance of androgen deprivation therapy such as, but not limited to, luteinizing hormone-releasing hormone (LHRH) agonist or LHRH antagonist, or a gonadotropin-releasing hormone (GnRH) agonist or GnRH antagonist. In some embodiments, the GnRH agonist is selected from the group consisting of leuprolide, buserelin, nafarelin, histrelin, goserelin, or deslorelin. In some embodiments the androgen deprivation therapy is leuprolide. In some embodiments the androgen deprivation therapy is goserelin. In some embodiments the androgen deprivation therapy is degarelix. In some embodiments the androgen deprivation therapy is relugolix.

In one embodiment, the cancer is metastatic castration-sensitive prostate cancer, and the subject has previously undergone an orchiectomy or a bilateral orchiectomy.

In one embodiment, the cancer is hormone sensitive prostate cancer, and the subject has previously undergone an orchiectomy or a bilateral orchiectomy but the cancer has since progressed.

In one embodiment of the present invention, the combination therapy is administered to a subject diagnosed with metastatic castration-sensitive prostate cancer, which subject has a prostate specific antigen level medically determined to be tumor-related.

### Dosage Forms and Regimens

Each therapeutic agent of the methods and combination therapies of the present invention may be administered either alone, or in a medicament (also referred to herein as a pharmaceutical composition) which comprises the therapeutic agent and one or more pharmaceutically acceptable carriers, excipients, or diluents, according to pharmaceutical practice.

As used herein, the term “combination therapy” refers to the administration of each therapeutic agent of the combination therapy of the invention, either alone or in a medicament, either sequentially, concurrently or simultaneously.

As used herein, the term “sequential” or “sequentially” refers to the administration of each therapeutic agent of the combination therapy of the invention, either alone or in a medicament, one after the other, wherein each therapeutic agent can be administered in any order. Sequential administration is particularly useful when the therapeutic agents in the combination therapy are in different dosage forms, for example, one agent is a tablet and another agent is a sterile liquid, and/or are administered according to different dosing schedules, for example, one agent is administered daily, and the second agent is administered less frequently such as weekly.

As used herein, the term “concurrently” refers to the administration of each therapeutic agent in the combination therapy of the invention, either alone or in separate medicaments, wherein the second therapeutic agent is administered immediately after the first therapeutic agent, but that the therapeutic agents can be administered in any order. In a preferred embodiment the therapeutic agents are administered concurrently.

As used herein, the term “simultaneous” refers to the administration of each therapeutic agent of the combination therapy of the invention in the same medicament.

In one embodiment of the present invention, talazoparib, or a pharmaceutically acceptable salt thereof, is administered before administration of the anti-androgen, or a pharmaceutically acceptable salt thereof.

In one embodiment of the present invention, talazoparib, or a pharmaceutically acceptable salt thereof, is administered before administration of the androgen receptor inhibitor, or a pharmaceutically acceptable salt thereof.

In one embodiment of the present invention, talazoparib, or a pharmaceutically acceptable salt thereof, is administered before administration of enzalutamide, or a pharmaceutically acceptable salt thereof.

In one embodiment of the present invention, the anti-androgen, or a pharmaceutically acceptable salt thereof, is administered before administration of talazoparib, or a pharmaceutically acceptable salt thereof.

In one embodiment of the present invention, the androgen receptor inhibitor, or a pharmaceutically acceptable salt thereof, is administered before administration of talazoparib, or a pharmaceutically acceptable salt thereof.

In one embodiment of the present invention, enzalutamide, or a pharmaceutically acceptable salt thereof, is administered before administration of talazoparib, or a pharmaceutically acceptable salt thereof.

In one embodiment of the present invention, talazoparib, or a pharmaceutically acceptable salt thereof, is administered concurrently with the anti-androgen, or a pharmaceutically acceptable salt thereof.

In one embodiment of the present invention, talazoparib, or a pharmaceutically acceptable salt thereof, is administered concurrently with the androgen receptor inhibitor, or a pharmaceutically acceptable salt thereof.

In one embodiment of the present invention, talazoparib, or a pharmaceutically acceptable salt thereof, is administered concurrently with enzalutamide, or a pharmaceutically acceptable salt thereof.

In one embodiment of the present invention, talazoparib, or a pharmaceutically acceptable salt thereof, is administered simultaneously with the anti-androgen, or a pharmaceutically acceptable salt thereof.

In one embodiment of the present invention, talazoparib, or a pharmaceutically acceptable salt thereof, is administered simultaneously with the androgen receptor inhibitor, or a pharmaceutically acceptable salt thereof.

In one embodiment of the present invention, talazoparib, or a pharmaceutically acceptable salt thereof, is administered simultaneously with enzalutamide, or a pharmaceutically acceptable salt thereof.

In one embodiment, talazoparib is talazoparib tosylate.

As will be understood by those skilled in the art, the combination therapy may be usefully administered to a subject during different stages of their treatment.

In one embodiment of the present invention, the combination therapy is administered to a subject who is previously untreated, i.e. is treatment naïve.

In one embodiment of the present invention, the combination therapy of the present invention is a first treatment option, i.e., first-line treatment, for a subject presenting with metastatic castration-sensitive prostate cancer.

In one embodiment of the present invention, the combination therapy is administered to a subject who has failed to achieve a sustained response after a prior therapy with a biotherapeutic or chemotherapeutic agent, i.e. is treatment experienced.

In one embodiment of the present invention, the combination therapy is administered to a subject who has previously received androgen deprivation therapy, such as, but not limited to, LHRH agonist or LHRH antagonist.

In one embodiment of the present invention, the combination therapy is administered to a subject who has previously received androgen deprivation therapy, such as, but not limited to, luteinizing hormone-releasing hormone (LHRH) agonist or LHRH antagonist, or a gonadotropin-releasing hormone (GnRH) agonist or a GnRH antagonist. In some embodiments, the GnRH agonist is selected from the group consisting of leuprolide, buserelin, nafarelin, histrelin, goserelin, or deslorelin.

In one embodiment of the present invention, the combination therapy is administered to a subject who has previously undergone a bilateral orchiectomy.

In one embodiment of the present invention, the combination therapy is administered to a subject who has previously received an anti-androgen or taxane.

In one embodiment of the present invention, the combination therapy is administered to a subject who has previously received an anti-androgen.

In one embodiment of the present invention, the combination therapy is administered to a subject who has previously received an androgen receptor inhibitor.

In one embodiment of the present invention, the combination therapy is administered to a subject who has previously received enzalutamide.

In one embodiment of the present invention, the combination therapy is administered to a subject who has previously received abiraterone acetate.

In one embodiment of the present invention, the combination therapy is administered to a subject who has previously received a PARP inhibitor.

In one embodiment of the present invention, the combination therapy is administered to a subject who has previously received androgen deprivation therapy, such as, but not limited to, luteinizing hormone-releasing hormone (LHRH) agonist and/or LHRH antagonist and/or a gonadotropin-releasing hormone (GnRH) agonist or GnRH antagonist; and/or has previously undergone a bilateral orchiectomy; and/or has previously received enzalutamide; and/or has previously received abiraterone but whose cancer has since progressed.

In one embodiment of the present invention, the combination therapy is administered to a subject who has previously received androgen deprivation therapy, such as, but not limited to, LHRH agonist and/or LHRH antagonist; and/or has previously undergone a bilateral orchiectomy; and/or has previously received enzalutamide; and/or has previously received abiraterone but whose cancer has since progressed.

In one embodiment of the present invention, the combination therapy is administered to a subject who has previously received androgen deprivation therapy, such as, but not limited to, luteinizing hormone-releasing hormone (LHRH) agonist or LHRH antagonist, or a gonadotropin-releasing hormone (GnRH) agonist or a GnRH antagonist, but whose cancer has since progressed.

In one embodiment of the present invention, the combination therapy is administered to a subject who has previously received androgen deprivation therapy, such as, but not limited to, LHRH agonist or LHRH antagonist, but whose cancer has since progressed.

In one embodiment of the present invention, the combination therapy is administered to a subject who has previously undergone a bilateral orchiectomy, but whose cancer has since progressed.

In one embodiment of the present invention, the combination therapy is administered to a subject who has previously received androgen receptor inhibitor, but whose cancer has since progressed.

In one embodiment of the present invention, the combination therapy is administered to a subject who has previously received enzalutamide, but whose cancer has since progressed.

In one embodiment of the present invention, the combination therapy is administered to a subject who has previously received abiraterone acetate, but whose cancer has since progressed.

In one embodiment of the present invention, the combination therapy is administered to a subject who has previously received a PARP inhibitor, but whose cancer has since progressed.

In one embodiment of the present invention, the combination therapy is administered to a subject diagnosed with prostate cancer, which subject has a prostate specific antigen level medically determined to be tumor-related.

In one embodiment of the present invention, the combination therapy is administered to a subject diagnosed with prostate cancer, which subject has a prostate specific antigen level of at least 2.0 ng/mL.

In one embodiment of the present invention, the combination therapy is administered to a subject diagnosed with prostate cancer, which subject has a prostate specific antigen level of at least 2.0 ng/mL, and which prostate specific antigen level has risen on at least two successive occasions at least 1 week apart.

In one embodiment of the present invention, the combination therapy is administered to a subject diagnosed with prostate cancer, which subject has a prostate specific antigen level which has doubled in ≤10 months.

In one embodiment of the present invention, the combination therapy is administered to a subject diagnosed with cancer, which cancer has developed resistance to treatment with an anti-androgen.

In one embodiment of the present invention, the combination therapy is administered to a subject diagnosed with cancer, which cancer has developed resistance to treatment with an anti-androgen.

In one embodiment of the present invention, the combination therapy is administered to a subject diagnosed with cancer, which cancer has developed resistance to treatment with an androgen receptor inhibitor.

In one embodiment of the present invention, the combination therapy is administered to a subject diagnosed with cancer, which cancer has developed resistance to treatment with a PARP inhibitor.

The combination therapy may be administered prior to or following surgery to remove a tumor and/or may be used prior to, during or after radiation therapy, and/or may be used prior to, during or after chemotherapy.

Administration of compounds of the invention may be effected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a therapeutic agent of the combination therapy of the present invention may be administered as a single bolus, as several divided doses administered over time, or the dose may be proportionally reduced or increased as indicated by the exigencies of the therapeutic situation. It may be particularly advantageous to formulate a therapeutic agent in a dosage unit form for ease of administration and uniformity of dosage. Dosage unit form, as used herein, refers to physically discrete units suited as unitary dosages for the mammalian subjects to be treated, each unit containing a predetermined quantity of active compound calculated to produce the desired therapeutic effect in association with the required pharmaceutical carrier. The specification for the dosage unit forms of the invention may be dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose may be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, taking into consideration factors such as the severity of the disorder or condition, the rate of administration, the disposition of the compound and the discretion of the prescribing physician. The dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the therapeutic agent are well-known in the relevant art and would be understood to be encompassed by the skilled artisan once provided the teachings disclosed herein.

In some embodiments, at least one of the therapeutic agents in the combination therapy is administered using the same dosage regimen (dose, frequency and duration of treatment) that is typically employed when the agent is used as a monotherapy for treating the same cancer. In other embodiments, the subject received a lower total amount of at least one of the therapeutic agents in the combination therapy than when the same agent is used as a monotherapy, for example a lower dose of therapeutic agent, a reduced frequency of dosing and/or a shorter duration of dosing.

An effective dosage of talazoparib, or a pharmaceutically acceptable salt thereof and preferably a tosylate thereof, is administered at a daily dosage of from about 0.1 mg to about 2 mg once a day, preferably from about 0.25 mg to about 1.5 mg once a day, and more preferably from about 0.5 mg to about 1.0 mg once a day. In an embodiment, talazoparib or a pharmaceutically acceptable salt thereof and preferably a tosylate thereof, is administered at a daily dosage of about 0.1 mg, about 0.25 mg, about 0.35 mg, about 0.5 mg, about 0.75 mg or about 1.0 mg once daily. In an embodiment, talazoparib or a pharmaceutically acceptable salt thereof and preferably a tosylate thereof, is administered at a daily dosage of about 0.1 mg, about 0.25 mg, about 0.35 mg, or about 0.5 mg once daily. In an embodiment, talazoparib or a pharmaceutically acceptable salt thereof and preferably a tosylate thereof, is administered at a daily dosage of about 0.25 mg, about 0.35 mg, or about 0.5 mg once daily. In an embodiment, talazoparib or a pharmaceutically acceptable salt thereof and preferably a tosylate thereof, is administered at a daily dosage of about 0.35 mg or about 0.5 mg once daily. In an embodiment, talazoparib or a pharmaceutically acceptable salt thereof and preferably a tosylate thereof, is administered at a daily dosage of about 0.5 mg, about 0.75 mg or about 1.0 mg once daily. In an embodiment, talazoparib or a pharmaceutically acceptable salt thereof and preferably a tosylate thereof, is administered at a daily dosage of about 0.1 mg once daily. In an embodiment, talazoparib or a pharmaceutically acceptable salt thereof and preferably a tosylate thereof, is administered at a daily dosage of about 0.25 mg once daily. In an embodiment, talazoparib or a pharmaceutically acceptable salt thereof and preferably a tosylate thereof, is administered at a daily dosage of about 0.35 mg once daily. In an embodiment, talazoparib or a pharmaceutically acceptable salt thereof and preferably a tosylate thereof, is administered at a daily dosage of about 0.5 mg once daily. In an embodiment, talazoparib or a pharmaceutically acceptable salt thereof and preferably a tosylate thereof, is administered at a daily dosage of about 0.75 mg once daily. In an embodiment, talazoparib or a pharmaceutically acceptable salt thereof and preferably a tosylate thereof, is administered at a daily dosage of about 1.0 mg once daily. Dosage amounts provided herein refer to the dose of the free base form of talazoparib, or are calculated as the free base equivalent of an administered talazoparib salt form. For example, a dosage or amount of talazoparib, such as about 0.5 mg, about 0.75 mg or about 1.0 mg refers to the free base equivalent. This dosage regimen may be adjusted to provide the optimal therapeutic response. For example, the dose may be proportionally reduced or increased as indicated by the exigencies of the therapeutic situation.

An effective dosage of an anti-androgen, or a pharmaceutically acceptable salt thereof, is in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

An effective dosage of an androgen receptor inhibitor, or a pharmaceutically acceptable salt thereof, is in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

In one embodiment the androgen receptor inhibitor is enzalutamide, which enzalutamide is dosed in accordance with the approved label with a daily dose of 160 mg once daily. Dosage adjustments of enzalutamide, in accordance with full prescribing information, such as if the enzalutamide is to be dosed in concomitantly with a strong CYP2C8 inhibitor then the dose of enzalutamide should be reduced in accordance with the full prescribing information, such as to 80 mg once daily; or alternatively if the enzalutamide is to be dosed concomitantly with a CYP3A4 inducer then the dose of enzalutamide should be increased in accordance with the full prescribing information, such as to 240 mg daily.

In an embodiment the anti-androgen is abiraterone acetate, which abiraterone acetate is dosed in accordance with the approved label with a daily dose of 1000 mg once daily in combination with prednisone 5 mg twice daily. Dosage adjustments of abiraterone acetate, in accordance with full prescribing information may be readily determined by one of ordinary skill in the art, such as if the abiraterone acetate is to be dosed concomitantly with a strong CYP3A4 inducer, then the dosage of abiraterone acetate may need to be increased for example to 1000 mg twice per day; if the abiraterone acetate is to be dosed concomitantly with a CYP2D6 substrate, then the dosage of abiraterone acetate may need to be reduced; if the abiraterone acetate is to be dosed to a subject or subject with baseline moderate hepatic impairment then the dose may need to be reduced, such as to 250 mg once daily; if the abiraterone acetate is to be dosed to a subject or subject who develops hepatotoxicity then the dose may need to be reduced, such as to 750 mg or 500 mg once daily.

Repetition of the administration or dosing regimens, or adjustment of the administration or dosing regimen may be conducted as necessary to achieve the desired treatment. A “continuous dosing schedule” as used herein is an administration or dosing regimen without dose interruptions, e.g. without days off treatment. Repetition of 21 or 28 day treatment cycles without dose interruptions between the treatment cycles is an example of a continuous dosing schedule. In an embodiment, the compounds of the combination of the present invention can be administered in a continuous dosing schedule.

In one embodiment of the present invention, talazoparib, or a pharmaceutically acceptable salt thereof, and the anti-androgen, or a pharmaceutically acceptable salt thereof, are dosed in amounts which together are effective in treating the cancer.

In one embodiment of the present invention, talazoparib, or a pharmaceutically acceptable salt thereof, and the androgen receptor inhibitor, or a pharmaceutically acceptable salt thereof, are dosed in amounts which together are effective in treating the cancer.

In one embodiment of the present invention, talazoparib, or a pharmaceutically acceptable salt thereof, and enzalutamide, or a pharmaceutically acceptable salt thereof, are dosed in amounts which together are effective in treating the cancer.

In one embodiment of the present invention talazoparib, or a pharmaceutically acceptable salt thereof, and the anti-androgen, or a pharmaceutically acceptable salt thereof, are dosed in a non-standard dosing regimen.

In one embodiment of the present invention, talazoparib, or a pharmaceutically acceptable salt thereof, and the androgen receptor inhibitor, or a pharmaceutically acceptable salt thereof, are dosed in a non-standard dosing regimen.

In one embodiment of the present invention, talazoparib, or a pharmaceutically acceptable salt thereof, and enzalutamide, or a pharmaceutically acceptable salt thereof, are dosed in a non-standard dosing regimen.

In one embodiment of the present invention talazoparib, or a pharmaceutically acceptable salt thereof, and the anti-androgen, or a pharmaceutically acceptable salt thereof, are dosed in a low dose regimen.

In one embodiment of the present invention talazoparib, or a pharmaceutically acceptable salt thereof, and the androgen receptor inhibitor, or a pharmaceutically acceptable salt thereof, are dosed in a low dose regimen.

In one embodiment of the present invention talazoparib, or a pharmaceutically acceptable salt thereof, and enzalutamide, or a pharmaceutically acceptable salt thereof, are dosed in a low dose regimen.

### Pharmaceutical Compositions and Routes of Administration

A “pharmaceutical composition” refers to a mixture of one or more of the therapeutic agents described herein, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients.

As used herein, a “pharmaceutically acceptable carrier” refers to a carrier or diluent that does not cause significant irritation to an organism and does not abrogate the biological activity and properties of the active compound or therapeutic agent.

In one embodiment, this invention relates to a pharmaceutical composition comprising talazoparib, or a pharmaceutically acceptable salt thereof, and an anti-androgen, or a pharmaceutically acceptable salt thereof, and a pharmaceutically acceptable carrier.

In one embodiment, this invention relates to a pharmaceutical composition comprising talazoparib, or a pharmaceutically acceptable salt thereof, and an androgen receptor inhibitor, or a pharmaceutically acceptable salt thereof, and a pharmaceutically acceptable carrier.

The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream, or for rectal administration as a suppository.

Exemplary parenteral administration forms include solutions or suspensions of an active compound in a sterile aqueous solution, for example, aqueous propylene glycol or dextrose solutions. Such dosage forms may be suitably buffered, if desired.

The pharmaceutical composition may be in unit dosage forms suitable for single administration of precise amounts.

Pharmaceutical compositions suitable for the delivery of the therapeutic agents of the combination therapies of the present invention, and methods for their preparation will be readily apparent to those skilled in the art. Such compositions and methods for their preparation may be found, for example, in ‘Remington's Pharmaceutical Sciences’, 19th Edition (Mack Publishing Company, 1995), the disclosure of which is incorporated herein by reference in its entirety.

Therapeutic agents of the combination therapies of the invention may be administered orally. Oral administration may involve swallowing, so that the therapeutic agent enters the gastrointestinal tract, or buccal or sublingual administration may be employed by which the therapeutic agent enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Therapeutic agents of the combination therapies of the present invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, the therapeutic agent may make up from 1 wt % to 80 wt % of the dosage form, more typically from 5 wt % to 60 wt % of the dosage form. In addition to the active agent, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant may comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets may contain up to about 80 wt % active agent, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles may be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

### Parenteral Administration

Therapeutic agents of the combination therapies of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of therapeutic agents used in the preparation of parenteral solutions may potentially be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus therapeutic agents of the combination therapies of the invention may potentially be formulated as a solid, semi-solid, or thixotropic liquid for administration as an implanted depot providing modified release of the active compound. Examples of such formulations include drug-coated stents and PGLA microspheres.

The therapeutic agents of the combination therapies of the invention may also potentially be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and micro needle or needle-free (e.g. Powderject™, Bioject™, etc.) injection. The disclosures of these references are incorporated herein by reference in their entireties.

Formulations for topical administration may be formulated to be immediate and/or modified release. Modified release formulations include delayed-, sustained-, pulsed-, controlled-, targeted and programmed release.

Therapeutic agents of the combination therapies of the invention may also potentially be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin.

The pressurized container, pump, spray, atomizer, or nebulizer may contain a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the compound may be micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the therapeutic agent, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the therapeutic agent per actuation and the actuation volume may vary from 1 μL to 100 μL. A typical formulation includes a therapeutic agent, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing a desired mount of the therapeutic agent. The overall daily dose may be administered in a single dose or, more usually, as divided doses throughout the day.

Therapeutic agents of the combination therapies of the invention may potentially be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

Therapeutic agents of the combination therapies of the invention may also potentially be administered directly to the eye or ear, typically in the form of drops of a micronized suspension or solution in isotonic, pH-adjusted, sterile saline. Other formulations suitable for ocular and aural administration may include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

In one embodiment, a pharmaceutical composition useful for the combination therapy of the present invention comprises only a single therapeutic agent, for example either talazoparib, or a pharmaceutically acceptable salt thereof; or an anti-androgen, or a pharmaceutically acceptable salt thereof; or an androgen receptor inhibitor, or a pharmaceutically acceptable salt thereof.

In another embodiment, a pharmaceutical composition useful for the combination therapy of the present invention comprises both talazoparib, or a pharmaceutically acceptable salt thereof, and an anti-androgen, or a pharmaceutically acceptable salt thereof.

In another embodiment, a pharmaceutical composition useful for the combination therapy of the present invention comprises both talazoparib, or a pharmaceutically acceptable salt thereof, and an androgen receptor inhibitor, or a pharmaceutically acceptable salt thereof.

### Kits

The therapeutic agents of the combination therapies of the present invention may conveniently be combined in the form of a kit suitable for coadministration of the compositions.

In one aspect, the present invention relates to a kit which comprises a first container, a second container and a package insert, wherein the first container comprises at least one dose of talazoparib, or a pharmaceutically acceptable salt thereof, the second container comprises at least one dose of an anti-androgen, or a pharmaceutically acceptable salt thereof, and the package insert comprises instructions for treating a subject for cancer using the medicaments.

In one aspect, the present invention relates to a kit which comprises a first container, a second container and a package insert, wherein the first container comprises at least one dose of talazoparib, or a pharmaceutically acceptable salt thereof, the second container comprises at least one dose of an androgen receptor inhibitor, or a pharmaceutically acceptable salt thereof, and the package insert comprises instructions for treating a subject for cancer using the medicaments.

In one embodiment, the kit of the present invention may comprise one or both of the active agents in the form of a pharmaceutical composition, which pharmaceutical composition comprises an active agent, or a pharmaceutically acceptable salt thereof, and a pharmaceutically acceptable carrier. The kit may contain means for separately retaining said compositions, such as a container, divided bottle, or divided foil packet. An example of such a kit is the familiar blister pack used for the packaging of tablets, capsules and the like.

The kit may be particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid. The kit may further comprise other materials that may be useful in administering the medicaments, such as diluents, filters, IV bags and lines, needles and syringes, and the like.

### Further Therapeutic Agents

In a further aspect, the methods and combination therapies of the present invention may additionally comprise administering further anti-cancer agents, such as anti-tumor agents, anti-angiogenesis agents, signal transduction inhibitors and antiproliferative agents, which amounts are together effective in treating said cancer. In some such embodiments, the anti-tumor agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, androgen deprivation therapy and anti-androgens.

In one embodiment of the methods and combination therapies of the present invention, the regimen includes a further active agent, wherein the further active agent is androgen deprivation therapy, such as an luteinizing hormone-releasing hormone (LHRH) agonist, an LHRH antagonist, or a gonadotropin-releasing hormone (GnRH) agonist or GnRH antagonist, including, but not limited to, leuprolide, buserelin, nafarelin, histrelin, goserelin, relugolix, degarelix, or deslorelin, and the like.

In one embodiment of the methods and combination therapies of the present invention, the regimen includes a further active agent, wherein the further active agent is androgen deprivation therapy, such as an LHRH agonist and the like.

In one embodiment the androgen deprivation therapy is a LHRH agonist.

In one embodiment the androgen deprivation therapy is a LHRH antagonist.

In one embodiment the androgen deprivation therapy is a GnRH agonist.

In one embodiment the androgen deprivation therapy is a GnRH antagonist.

In one embodiment the androgen deprivation therapy is selected from the group consisting of leuprolide (also known as leuprorelin, for example Lupron or Eligardor Viadur and the like); buserelin (for example Suprefact); gonadorelin; goserelin (for example Zoladex); histrelin (for example Vantas); nafarelin; triptorelin (for example Trelstar); deslorelin; fertirelin; abarelix (for example Plenaxis); cetrorelix; degarelix (for example Firmagon); ganirelix; ozarelix; elagolix (for example Orilissa); relugolix; and linzagolix.

In one embodiment the androgen deprivation therapy is selected from the group consisting of leuprolide, goserelin, degarelix and relugolix.

In one embodiment the androgen deprivation therapy is leuprolide. In some embodiments the leuprolide is administered intramuscularly at a dose of about 7.5 mg every month, or about 22.5 mg every three months, or about 30 mg every four months.

In one embodiment the androgen deprivation therapy is leuprolide. In some embodiments the leuprolide is administered subcutaneously at a dose of about 7.5 mg every month, or about 22.5 mg every three months, or about 30 mg every four months, or about 45 mg every six months, or about 65 mg every 12 months.

In one embodiment the androgen deprivation therapy is goserelin. In some embodiments the goserelin is administered subcutaneously at a dose of about 3.6 mg every month, or about 10.8 mg every three months.

In one embodiment the androgen deprivation therapy is degarelix. In some embodiments the degarelix is administered intramuscularly at an initial dose of about 240 mg, which initial dose may be optionally divided into several smaller doses, for example 2 doses of about 120 mg, followed by a maintenance dose of about 80 mg every month.

In one embodiment the androgen deprivation therapy is relugolix. In some embodiments the relugolix is administered orally at an initial dose of about 360 mg, which initial dose may be optionally divided into several smaller doses, for example 3 doses of about 120 mg, followed by a daily maintenance dose of about 120 mg.

In one embodiment of the methods and combination therapies of the present invention, the regimen includes a further active agent, wherein the further active agent is etoposide. In some embodiments, the etoposide is administered intravenously in accordance with the approved label, for example at a dose of from 50 to 100 mg/m² once a day on days 1 to 5; or from 5 to 100 mg/m² once a day on days 1, 3 and 5. In one example etoposide may be administered at a dose from 80 to 120 mg/m², on days 1, 2 and 3 of each 21-day cycle for 1, 2, 3, 4, 5 or 6 cycles.

The following abbreviations are used throughout and in the Example below: “ANC” means absolute neutrophil count; “AST” means aspartate aminotransferase; “ALT” means alanine aminotransferase; “BPI-SF” means Brief Pain Inventory Short Form; “CTCAE” means Common Terminology Criteria for Adverse Events; “eGFR” means estimated glomerular filtration rate; EORTC QLQ-PR25” means European Organisation for Research and Treatment of Cancer disease-specific urinary symptoms questionnaire; “EORTC QLQ-C30” means European Organisation for Research and Treatment of Cancer cancer-specific global health questionnaire; “MDRD” means modification of diet in renal disease; “NCI” means National Cancer Institute; “PCWG3” means Prostate Cancer Working Group; and “ULN” means upper limit of normal.

### Example 1: A Phase 3, Randomized, Double-Blind, Study of Talazoparib with Enzalutamide Versus Placebo with Enzalutamide in Men with DDR Gene Mutated Metastatic Castration-Sensitive Prostate Cancer

### Rationale and Objectives

The purpose of this clinical study is to evaluate the safety and efficacy of talazoparib in combination with enzalutamide compared with placebo in combination with enzalutamide in subjects with DDR-deficient mCSPC.

### Primary Objective:

To demonstrate that talazoparib in combination with enzalutamide is superior to placebo in combination with enzalutamide in prolonging investigator-assessed radiographic progression-free survival (rPFS), in subjects with mCSPC harboring DDR deficiencies.

### Secondary Objectives:

To demonstrate that talazoparib in combination with enzalutamide is superior to placebo in combination with enzalutamide in prolonging OS in subjects with mCSPC harboring DDR deficiencies.
To evaluate antitumor activity in subjects with mCSPC harboring DDR deficiencies with respect to the following:
   Objective response in measurable soft tissue disease;
   Duration of response in measurable soft tissue disease;
   PSA response;
   Time to PSA progression;
   Time to initiation of antineoplastic therapy;
   Time to first symptomatic skeletal event;
   Opiate use for prostate cancer pain.
To evaluate safety of talazoparib and enzalutamide administered in combination.
To evaluate the pharmacokinetic(s) (PK) of talazoparib and enzalutamide (and its N-desmethyl metabolite) when dosed in combination.
To evaluate the following subject reported outcomes in each treatment arm in subjects with mCSPC harboring DDR deficiencies:
   Pain symptoms;
   Cancer specific global health status/quality of life (QoL), functioning, and symptoms outcomes;
   General health status.
To assess the relationship between circulating tumor deoxyribonucleic acid (ctDNA) burden and outcome.

### Methods

### Clinical Trial Design:

This is a randomized, double blind, and placebo-controlled Phase 3 study to evaluate the safety and efficacy of talazoparib in combination with enzalutamide versus placebo in combination with enzalutamide in subjects with mCSPC.

Genomic screening of a peripheral blood sample during the prescreening and prior to randomization is required for eligibility. Mutational status for subjects will be determined by testing for the presence of mutations in defined DDR genes likely to sensitize to PARP inhibition using the FoundationOne® Liquid CDx test that includes a DDR gene panel consisting of 12 genes, including ATM, ATR, BRCA1, BRCA2, CDK12, CHEK2, FANCA, MLH1, MRE11A, NBN, PALB2, and RAD51C. Historical FoundationOne® Liquid CDx results may be considered as an alternative to the blood sample and prior (i.e., historical) or de novo testing results of tumor tissue performed using the FoundationOne® test may be considered.

### Treatment:

The study has 5 periods: prescreening, screening, double-blind treatment, safety follow-up, and long-term follow-up.

Eligible subjects will be randomly assigned to either of 2 treatment groups as follows:
Talazoparib in combination with enzalutamide.
Placebo capsules identical in appearance to talazoparib capsules in combination with enzalutamide.

Randomization (1:1) to talazoparib or placebo will proceed based on the following stratification factors:
De novo mCSPC vs relapsed mCSPC.
High volume disease vs low volume disease.
   High volume disease is defined as the presence of visceral metastases or ≥4 bone lesions with ≥1 beyond the vertebral bodies and pelvis.
BRCA vs non-BRCA mutational status.

Talazoparib or identical placebo treatment will be blinded. Enzalutamide will be open label at a dose of 160 mg once daily. The dose of talazoparib to be given in combination with enzalutamide is 0.5 mg once daily. Subjects with moderate renal impairment (eGFR 30-59 mL/min/1.73 m² by the MDRD equation) at screening may be enrolled and the talazoparib dose will be 0.35 mg once daily.

Subjects will take all study intervention daily (at approximately the same time) and undergo the assessments until permanent discontinuation criteria are met. Dose modifications are allowed.

For all subjects, study intervention (including enzalutamide) should continue until radiographic progression is determined by investigator (unless in the opinion of the investigator the subject is still deriving benefit at this time), an adverse event (AE) leading to permanent study intervention discontinuation, subject decision to discontinue study intervention, or death.

### Inclusion Criteria:

1. Male subjects at least 18 years of age at screening (20 years for Japan; 19 years in the Republic of Korea).
2. Histologically or cytologically confirmed adenocarcinoma of the prostate without small cell or signet cell features. Alternatively, histologically or cytologically confirmed adenocarcinoma of the prostate without neuroendocrine differentiation, small cell or signet cell features. If the subject does not have a prior histological diagnosis, a baseline de novo biopsy must be used to confirm the diagnosis and may also be used to support biomarker analysis.
3. Confirmation of DDR gene mutation status by prospective or historical analysis (with sponsor pre-approval) of blood (liquid biopsy) and/or de novo or archival tumor tissue using FoundationOne® Liquid CDx or FoundationOne® CDx.
4. Willing to provide tumor tissue when available (de novo or archived) for retrospective molecular profiling analysis, if not already provided as part of inclusion criterion 3.
5. Unless prohibited by local regulations or ethics committee decision, consent to a saliva sample collection for retrospective sequencing of the same DDR genes tested on tumor tissue and blood (liquid biopsy), or a subset thereof, and to serve as a germline control in identifying tumor mutations.
6. Surgically or medically castrated, with serum testosterone less or equal to 50 ng/dL (less or equal to 1.73 nmol/L) at screening. Ongoing ADT with a GnRH agonist or antagonist for subjects who have not undergone bilateral orchiectomy must be initiated at least 4 weeks before randomization and must continue throughout the study. Alternatively, surgical or medical castration, with serum testosterone less or equal to 50 ng/dL (less or equal to 1.73 nmol/L) at screening, is not an inclusion criteria; and ongoing ADT with a GnRH agonist or antagonist for subjects who have not undergone bilateral orchiectomy must be initiated before randomization and must continue throughout the study.
7. Metastatic prostate cancer documented by positive bone scan (for bone disease) or metastatic lesions on CT or MRI scan (for soft tissue). Subjects whose disease spread is limited to regional pelvic lymph nodes are not eligible. Note: a finding of superscan at baseline is exclusionary.
8. Prior docetaxel therapy for mCSPC (up to 6 cycles) is allowed (must be completed 2 weeks prior to randomization and all toxicities from treatment have resolved). Alternatively, prior docetaxel therapy for mCSPC (up to 6 cycles) is not permitted.
9. Treatment with estrogens, cyproterone acetate, or first-generation anti-androgens is allowed until randomization.
10. Other prior therapy allowed for mCSPC; ≤6 months of ADT and ≤3 months of approved NHT in mCSPC (ie, abiraterone+prednisone, apalutamide, or enzalutamide), if required prior to randomization. Alternatively, other prior therapy allowed for mCSPC; ≤3 months of ADT (chemical or surgical) with or without approved NHT in mCSPC (ie, abiraterone+prednisone, apalutamide, or enzalutamide), if required prior to randomization, with no radiographic evidence of disease progression or rising PSA levels prior to Day 1. NOTE: Radical prostatectomy or definitive radiotherapy to the primary tumor for metastatic castration-sensitive prostate cancer with curative intent is not permitted.
11. Subject may have received palliative radiation or surgery for symptomatic control secondary to prostate cancer, which should have been completed at least 2 weeks prior to randomization.
12. ECOG performance status 0 or 1.
13. Adequate organ function within 28 days before the first study treatment on Day 1, defined by the following:
ANC ≥1500/μL, platelets ≥100,000/μL, or hemoglobin ≥9 g/dL (may not have received growth factors or blood transfusions within 14 days before obtaining the hematology laboratory tests at screening).
Total serum bilirubin <1.5×ULN (<3×ULN for subjects with documented Gilbert syndrome or for whom indirect bilirubin concentrations suggest an extrahepatic source of elevation).
AST or ALT <2.5×ULN (<5×ULN if liver function abnormalities are due to hepatic metastasis).
Albumin >2.8 g/dL.
eGFR ≥30 mL/min/1.73 m2 by the MDRD equation.
   14. Sexually active subjects that in the opinion of the investigator are capable of ejaculating, must agree to use a condom when having sex with a partner (female or male) from the time of the first dose of study treatment through 4 months after last dose of study treatment (or, if talazoparib/placebo has been stopped more than a month earlier than enzalutamide, through 3 months after last dose of enzalutamide). Must also agree for female partner of childbearing potential to use an additional highly effective form of contraception from the time of the first dose of study treatment through 4 months after last dose of study treatment.
   15. Must agree not to donate sperm from the first dose of study treatment to 4 months after the last dose of study treatment (or, if talazoparib/placebo has been stopped more than a month earlier than enzalutamide, through 3 months after last dose of enzalutamide).
   16. Subjects who are willing and able to comply with all scheduled visits, treatment plan, laboratory tests, lifestyle considerations, and other study procedures, including being able to manage electronic diaries. The PRO assessments are not required to be completed if a patient does not understand the language(s) available for a specific questionnaire and/or cannot complete the specific questionnaire independently.
   17. Capable of giving signed informed consent.

### Exclusion Criteria:

1. Other acute or chronic medical (concurrent disease, infection, including chronic stable HIV, HBV, or HCV infection, or co-morbidity) or psychiatric condition including recent (within the past year) or active suicidal ideation/behavior or laboratory abnormality that interferes with a subject's ability to participate in the study, may increase the risk of associated with study participation or study treatment administration, or may interfere with the interpretation of study results, and, in the investigator's judgment, make the subject inappropriate for entry into the study. HIV/HBV/HCV testing is not required unless mandated by local health authority.
2. History of seizure or any condition (as assessed by investigator) that may predispose to seizure (eg, prior cortical stroke, significant brain trauma), including any history of loss of consciousness or transient ischemic attack within 12 months of randomization.
3. Major surgery (as defined by the investigator) within 2 weeks before randomization.
4. Known or suspected brain metastasis or active leptomeningeal disease.
5. Symptomatic or impending spinal cord compression or cauda equina syndrome.
6. Any history of myelodysplastic syndrome (MDS), acute myeloid leukemia (AML), or prior malignancy except for the following:
Carcinoma in situ or non-melanoma skin cancer.
A cancer diagnosed and treated ≥3 years before randomization with no subsequent evidence of recurrence.
American Joint Committee on Cancer Stage 0 or Stage 1 cancer <3 years before randomization that has a remote probability of recurrence in the opinion of the investigator and the sponsor.
   7. In the opinion of the investigator, any clinically significant gastrointestinal disorder affecting absorption.
   8. Clinically significant cardiovascular disease, including any of the following:
Myocardial infarction or symptomatic cardiac ischemia within 6 months before randomization.
Congestive heart failure New York Heart Association class III or IV.
History of clinically significant ventricular arrhythmias (eg, sustained ventricular tachycardia, ventricular fibrillation, torsade de pointes) within 1 year before screening.
History of Mobitz II second degree or third-degree heart block unless a permanent pacemaker is in place.
Hypotension as indicated by systolic blood pressure <86 mm Hg at screening.
Bradycardia as indicated by a heart rate of <45 beats per minute on the screening electrocardiogram.
Uncontrolled hypertension as indicated by systolic blood pressure >170 mm Hg or diastolic blood pressure >105 mm Hg at screening. However, subjects can be rescreened after adequate control of blood pressure is achieved.
   9. Active COVID-19 infection detected by viral test or based on clinical diagnosis (as assessed by investigator). Asymptomatic subjects with no active COVID-19 infection detected but positive antibody tests, indicating past infection are allowed.
   10. Prior ADT in the adjuvant/neoadjuvant setting, where the completion of ADT was less than 12 months prior to randomization and the total duration of ADT exceeded 36 months.
   11. Subject received treatment with systemic glucocorticoids greater than the equivalent of 10 mg per day of prednisone within 4 weeks prior to randomization, intended for the treatment of prostate cancer.
   12. Any previous treatment with DNA-damaging cytotoxic chemotherapy (ie, platinum-based therapy) within 5 years prior to randomization, except for indications other than prostate cancer.
   13. Prior treatment with a PARP inhibitor or known or possible hypersensitivity to enzalutamide, any of enzalutamide capsule excipients or to any talazoparib/placebo capsule excipients.
   14. Prior treatment of mCSPC with docetaxel. Alternatively, prior treatment of mCSPC with docetaxel is not an exclusion criteria.
   15. Prior treatment in any setting with NHT, except as described in Inclusion Criterion #10. Alternatively, prior treatment in any setting with NHT, except ≤3 months of ADT (chemical or surgical) with or without approved NHT in mCSPC (ie, abiraterone+prednisone, apalutamide, or enzalutamide), if required prior to randomization
   16. Current use of potent P-glycoprotein (P-gp) inhibitors within 7 days prior to randomization.
   17. Treatment with any investigational study intervention within 4 weeks before randomization. Exception: COVID-19 vaccines authorized under an emergency use authorization (or equivalent) can be administered without a washout period.
   18. Baseline 12-lead electrocardiogram (ECG) that demonstrates clinically relevant abnormalities that may affect subject safety or interpretation of study results (eg, QTcF interval >470 msec, complete LBBB, signs of an acute or indeterminate age myocardial infarction, ST-T interval changes suggestive of myocardial ischemia, second or third degree AV block, or serious bradyarrhythmias or tachyarrhythmias). If the baseline uncorrected QT interval is >470 msec, this interval should be rate-corrected using the Fridericia method and the resulting QTcF should be used for decision making and reporting. If QTc exceeds 470 msec, or QRS exceeds 120 msec, the ECG should be repeated 2 more times and the average of the 3 QTc or QRS values should be used to determine the subject's eligibility. Computer-interpreted ECGs should be overread by a physician experienced in reading ECGs before excluding subjects.
   19. Investigator site staff or Sponsor employees directly involved in the conduct of the study, site staff otherwise supervised by the investigator, and their respective family members.

### Primary Endpoints:

Investigator-assessed radiological Progression-Free Survival (rPFS) per RECIST 1.1 (soft tissue disease) and PCWG3 (bone disease) in subjects with mCSPC harboring DDR deficiencies. rPFS is defined as the time from the date of randomization to first objective evidence of radiographic progression or death, whichever occurs first.

### Secondary Endpoints:

Overall Survival (OS) in subjects with mCSPC harboring DDR deficiencies (alpha-protected). OS is defined as the time from randomization to the date of death due to any cause.
Objective response in measurable soft tissue disease: Proportion of subjects with measurable soft tissue disease at baseline with an objective response per RECIST 1.1.
Duration of response in measurable soft tissue disease: Duration of responses in patients with measurable soft tissue disease at baseline per RECIST 1.1
Prostate Specific Antigen (PSA) response: Proportion of subjects with PSA response greater than or equal to 50% in subjects with detectable PSA values at baseline.
Time to PSA progression, which is defined as the time from baseline to PSA progression.
Time to initiation of antineoplastic therapy, which is defined as the time from randomization to initiation of antineoplastic therapy.
Time to first symptomatic skeletal event, which is defined as the time from randomization to first symptomatic skeletal event (symptomatic fractures, spinal cord compression, surgery or radiation to the bone whichever is first).
Time to opiate use for prostate cancer pain, which is defined as the time from randomization to opiate use for prostate cancer pain.
Incidence of AEs characterized by type, severity (graded by NCI CTCAE version 4.03), timing, seriousness and relationship to study intervention.
Pharmacokinetic assessment of enzalutamide and its metabolite: plasma concentrations of talazoparib, enzalutamide and its N-desmethyl metabolite.
Patient-reported outcomes in pain symptoms—change from baseline: Change from baseline in subject-reported pain symptoms per Brief Pain Inventory Short Form (BPI-SF).
Patient-reported outcomes in cancer specific general health status—change from baseline: Change from baseline in subject-reported general health status per EQ-5D-5L.
Patient-reported outcomes in cancer specific global health status/QoL—change from baseline: Change from baseline in subject-reported cancer specific global health status/QoL, functioning, and symptoms per EORTC QLQ-C30.
Patient-reported outcomes in pain symptoms—time to deterioration, which is defined as the time to deterioration in subject-reported pain symptoms per BPI-SF.
Patient-reported outcomes in cancer specific global health status/QoL—time to definitive deterioration: Time to definitive deterioration in subject-reported global health status/QoL per EORTC QLQ-C30.
Patient-reported outcomes in cancer specific symptoms—time to definitive deterioration: Time to definitive deterioration in subject-reported disease specific urinary symptoms per EORTC QLQ-PR25.
Patient-reported outcome: cancer specific functioning, and symptoms—change from baseline Change from baseline in PGI-S.
Relationship between ctDNA burden and outcome: ctDNA burden at baseline and on study, as assessed using FoundationOne® liquid or another suitable validated assay.

All publications and patent applications cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

What is claimed is:

1. A method of treating metastatic castration-sensitive prostate cancer in a subject in need thereof, wherein the subject has been identified as having at least one DNA damage repair gene mutation, comprising administering to the subject a combination therapy which comprises talazoparib, or a pharmaceutically acceptable salt thereof, and an anti-androgen, or a pharmaceutically acceptable salt thereof.

2. A method of treating metastatic castration-sensitive prostate cancer in a subject in need thereof, comprising a) detecting at least one DNA damage repair gene mutation from a biopsy of the metastatic cancer or a peripheral blood sample from the subject; and b) administering to the subject a combination therapy which comprises talazoparib, or a pharmaceutically acceptable salt thereof, and an anti-androgen, or a pharmaceutically acceptable salt thereof.

3. The method of claim 1 or claim 2, wherein the at least one DNA damage repair gene mutation is selected from the group consisting of ATM, ATR, BRCA1, BRCA2, CDK12, CHEK2, FANCA, MLH1, MRE11A, NBN, PALB2, and RAD51C.

4. The method of any one of claims 1-3, wherein the subject is treatment naive.

5. The method of any one of claims 1-4, wherein the talazoparib, or pharmaceutically acceptable salt thereof, is talazoparib tosylate.

6. The method of any one of claims 1-5, wherein the anti-androgen, or a pharmaceutically acceptable salt thereof, is an androgen receptor inhibitor, or a pharmaceutically acceptable salt thereof.

7. The method of claim 6, wherein the anti-androgen is selected from the group consisting of:
abiraterone acetate,
enzalutamide,
N-desmethyl enzalutamide,
darolutamide, and
apalutamide;
or a pharmaceutically acceptable salt thereof.

8. The method of claim 6, wherein the anti-androgen is enzalutamide, or a pharmaceutically acceptable salt thereof.

9. The method of any one of claims 1-8, wherein the talazoparib, or pharmaceutically acceptable salt thereof, is administered at a daily dosage of about 0.35 mg or about 0.5 mg once daily and the enzalutamide, or a pharmaceutically acceptable salt thereof, is administered at a daily dosage of about 160 mg.

10. The method of any one of claims 1-8, wherein the talazoparib, or pharmaceutically acceptable salt thereof, is administered at a daily dosage of about 0.35 mg once daily and the enzalutamide, or a pharmaceutically acceptable salt thereof, is administered at a daily dosage of about 160 mg.

11. The method of any one of claims 1-8, wherein the talazoparib, or pharmaceutically acceptable salt thereof, is administered at a daily dosage of about 0.5 mg once daily and the enzalutamide, or a pharmaceutically acceptable salt thereof, is administered at a daily dosage of about 160 mg.

12. The method of any one of claims 1-8, wherein the talazoparib, or pharmaceutically acceptable salt thereof, is administered at a daily dosage of about 0.35 mg once daily.

13. The method of any one of claims 1-8, wherein the talazoparib, or pharmaceutically acceptable salt thereof, is administered at a daily dosage of about 0.5 mg once daily.

14. The method of any one of claims 1-8, wherein the enzalutamide, or a pharmaceutically acceptable salt thereof is administered at a daily dosage of about 160 mg.

15. The method of any one of claims 1-8, wherein the talazoparib, or pharmaceutically acceptable salt thereof, and the anti-androgen, or pharmaceutically acceptable salt thereof, are each in an amount that is together effective in treating metastatic castration-sensitive prostate cancer.

16. The method of any one of claims 1-8, wherein the talazoparib, or pharmaceutically acceptable salt thereof, and the anti-androgen, or a pharmaceutically acceptable salt thereof, are administered concurrently.

17. The method of any one of claims 1-8, wherein the method comprises administering a further anti-cancer agent.

18. The method of any one of claims 1-8, wherein the further anti-cancer agent is selected from the group consisting of an anti-tumor agent, an anti-angiogenesis agent, a signal transduction inhibitor, an antiproliferative agent, and androgen deprivation therapy.

19. The method of claim 18, wherein the androgen deprivation therapy is selected from the group consisting of a luteinizing hormone-releasing hormone agonist, a luteinizing hormone-releasing hormone antagonist, a gonadotropin releasing hormone agonist, a gonadotropin releasing hormone antagonist, and bilateral orchiectomy.

20. The method of any of the proceeding claims, wherein the subject is a human.
